(19) United States
(12) Patent Application Publication
(10) Pub. No.: US 2026/0315049 A1
(43) Pub. Date: Oct. 15, 2026

(54) ERGONOMIC SURGICAL DEVICE

(71) Applicant: THE TRUSTEES OF COLUMBIA UNIVERSITY IN THE CITY OF NEW YORK, New York, NY (US)

(72) Inventor: Arvind NANDAKUMAR, Baton Rouge, LA (US)

(21) Appl. No.: 19/630,642

(22) Filed: Mar. 27, 2026

Related U.S. Application Data
(63) Continuation of application No. PCT/US2024/049209, filed on Sep. 30, 2024.
(60) Provisional application No. 63/585,999, filed on Sep. 28, 2023.
(60) Provisional application No. 63/651,503, filed on May 24, 2024.

(51) Int. Cl. A61B 17/00

(52) U.S. Cl. CPC A61B 17/00; A61B 2017/00017; A61B 2017/00398; A61B 2017/00734; A61B 2017/00862

(57) ABSTRACT

A surgical device, comprising: a handpiece, the handpiece comprising a touch sensor; a shaft extending from the handpiece and having a distal end; a surgical element disposed generally at the distal end of the shaft; and an actuator, the actuator being controllable by the touch sensor, and the actuator modulating movement of the surgical element. A surgical device, comprising: a handpiece; a surgical element; and a control train configured to control a movement of the surgical element in response to a user's touch on a touch sensor of the control train. A method, comprising: in response to a touch on a touch sensor of a surgical device, controlling any one or more of a direction of motion or a degree of motion of a surgical element of the surgical device.

### RELATED APPLICATIONS

The present application is a continuation application of PCT/US2024/049209, “Ergonomic Surgical Device” (filed Sep. 30, 2024); which claims priority to and the benefit of U.S. patent application No. 63/585,999, “Ergonomic Surgical Device” (filed Sep. 28, 2023) and U.S. patent application No. 63/651,503, “Ergonomic Surgical Device” (filed May 24, 2024). All foregoing applications are incorporated herein by reference in their entireties for any and all purposes.

### TECHNICAL FIELD

The present disclosure relates to the field of surgical devices and to the field of capacitive instrument controls.

### BACKGROUND

Existing handheld surgical instruments—for example, laparoscopic devices—can be uncomfortable for users, and extended usage of these instruments can lead to pain and even hand injuries for surgeons and increase the potential for surgical errors. Although efforts have been made to enhance the ergonomics of surgical handles, the majority of these improvements depend on manual control. Although some motorized and robot-assisted surgical devices exist, such devices nonetheless employ mechanical buttons and dials, and robot-assisted instruments are costly. Accordingly, there is a long-felt need in the art for surgical devices with improved ergonomics.

### SUMMARY

In meeting the described long-felt needs, the disclosed technology addresses shortcomings in typical surgical graspers by providing a touch-controlled surgical instrument. Through touch sensors, this technology addresses the fatigue issue present in existing products, and the disclosed technology also offers precision control by enabling surgeons to translate touch gestures into accurate movements during surgical procedures. As explained herein, the disclosed technology can include devices that feature a touch sensor operable by a tap to increase and decrease resolution of a sliding mechanism of the device.

In one aspect, the present disclosure provides a surgical device, comprising: a handpiece, the handpiece comprising a touch sensor; a shaft extending from the handpiece and having a distal end; a surgical element disposed generally at the distal end of the shaft; and an actuator, the actuator being controllable by the touch sensor, and the actuator modulating movement of the surgical element.

Also provided is a surgical device, comprising: a handpiece; a surgical element; and a control train configured to control a movement of the surgical element in response to a user's touch on a touch sensor of the control train.

Further provided is a method, comprising: in response to a touch on a touch sensor of a surgical device, controlling any one or more of a direction of motion or a degree of motion of a surgical element of the surgical device.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1-25 provide example, non-limiting embodiments of the disclosed technology.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,”“has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments of aspects can be combined with any part or parts of any one or more other embodiments or aspects.

Various non-limiting, exemplary embodiments of the disclosed technology are provided below. It should be understood that these embodiments are exemplary only and that the disclosed technology is not confined to these embodiments. As an example, the sensors on the disclosed devices need not necessarily be placed where the sensors are shown in the attached figures and described examples.

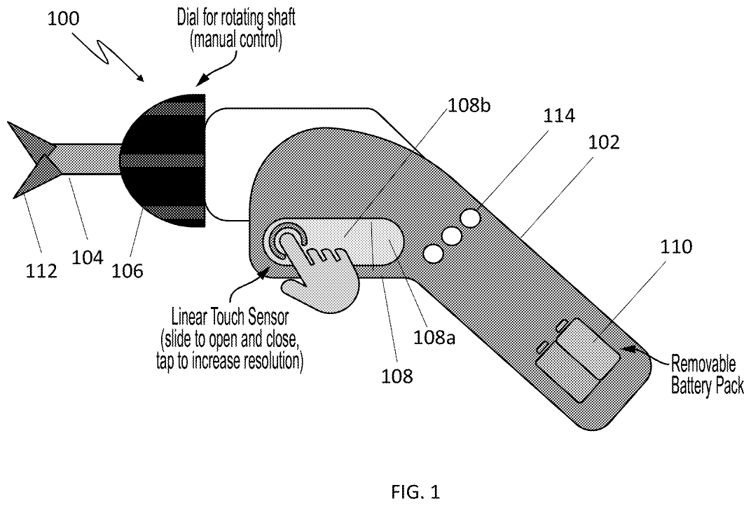

FIG. 1 provides an illustration of an exemplary embodiment of the disclosed technology. As shown, surgical device 100 can include handpiece 102, which can further comprise touch sensor 108. Handpiece 102 can be adjustable, for example, such that the angle of the handle around which a user's hand wraps can be varied. Handpiece 102 can also be configured to allow a user's hand to remain in a comfortable position during use of device 100.

Touch sensor 108 can be positioned such that it is actuated by the index finger of the user. This is not, however, a requirement, as touch sensor 108 can be positioned so that the sensor is actuated by the user's thumb and/or another of the user's digits. The touch sensor can be responsive to slide touches, tap touches, persistent touches, and the like. Touch sensor 108 need not necessarily be located where it is shown in FIG. 1, however. As an example, a sensor can be located such that it is on the top of the device when the device is being used. A sensor can also be located on the side of the device, on the bottom of the device, the front of the device, even on the back or rear portion of the device. A sensor can, in some embodiments, be moveable or re-mountable so as to accommodate different hand or finger sizes, as well as to accommodate different user preferences. A device can, for example, include multiple sensors but be configured such that one or more of the sensors is inactive during use. This can be useful, for instance, to allow a right-handed user to use the same device as a left-handed user and vice versa. In such a situation, a device can include sensors on the right and left sides, and the left sensor can be deactivated while the right sensor remains active. This can in turn facilitate use of the device by a user who prefers to control the device with their right hand instead of their left.

Device 100 can also include shaft 104, which can extend away from handpiece 102. Surgical element 112 can be located at an end (for example, the distal end) of shaft 104. As explained elsewhere herein, a surgical element can comprise any one or more of a jaw, a blade, an arm, and a needle. A device can include, of course, a plurality of surgical elements, for example, opposing jaws.

Device 100 can be configured such that touches on touch sensor 108 actuate surgical element 112. As an example, a sliding motion along touch sensor 108 can act to close and/or open jaw surgical elements. Likewise, a sliding motion along touch sensor 108 can act to close and/or open surgical elements that are blades, for example, in the manner of a scissors. It should be understood that device 100 can be configured such that a tap and/or a persistent touch can actuate surgical element 112. As an example, a tap and/or a persistent touch on sensor 108 can act to close and/or open jaw surgical elements. Likewise, a tap and/or a persistent touch on touch sensor 108 can act to close and/or open surgical elements that are blades, for example, in the manner of scissors.

Device 100 can be configured such that a tap—which can be a brief tap or even a persistent tap—on touch sensor acts to control the degree (also termed “resolution”) of movement of surgical element 112. For example, moving one's finger along the distance touch sensor 108 while the device is in a first state can fully close surgical element 112.

Device 100, can, however, be configured such that a user's tap or taps on touch sensor 108 can place the device in a state in which moving one's finger along the distance touch sensor 108 while the device is in a first state closes surgical element 112 only half way. Device 100, can, however, be configured such that a user's tap or taps on touch sensor 108 can place the device in a state in which moving one's finger along the distance touch sensor 108 while the device is in a first state closes surgical element 112 only one quarter of the full way. In this way, a user can control not only the general movement of surgical element 112, but the user can also control the degree of movement of movable element 112. It should be understood that device 100 can include a single touch sensor 108 that allows a user to control the general movement (for example, movement of jaws from an open state to a closed state) of surgical element 112 and also the degree of movement of surgical element 112 from that single touch sensor.

As shown, touch sensor 108 can—but does not have to—comprise a first region 108a and a second region 108b. In some embodiments, touching region 108a effects a different motion of surgical element 112 than does touching region 108b. For example, touching region 108a can open surgical element 112, and touching region 108b can close surgical element 112. As another example, a sliding touch along region 108b can open or close surgical element 112, and a tap and on region 108a can modulate the degree and/or speed of opening or closing of surgical element 112. In this way, different regions of touch sensor 108 can be associated with different kinds of control of surgical device 100.

Device 100 can also, in some embodiments, include multiple distinct touch sensors, for example a first touch sensor that controls the general movement of surgical element 112 and also a second touch sensor that controls the degree of movement of surgical element 112. As an example, device 100 can be configured such that the general movement (for example, opening and/or closing) of surgical element 112 is controlled by touches of a touch sensor by a user's index finger, and the degree and/or speed of movement of surgical element 112 is controlled by touches of a touch sensor by a user's thumb. Different actions can be assigned to different sensors, which can be useful to accommodate user preferences.

Touch sensor 108 can provide haptic feedback to a user. Such feedback can be based, for example, on a position and/or state of surgical element 112. Such feedback can be based, for example, on contact (or lack of contact) between surgical element 112 and tissue or another object; feedback can be related to a stepper motor and/or a rotary encoder.

Feedback can also be related to a resolution setting; as an example a user can receive increased vibration frequency for a larger-movement resolution and a reduced vibration frequency for a smaller-movement resolution. Haptic feedback can also be delivered to a user through other portions of the device, for example, through handpiece 102. Without being bound to any particular theory or embodiment, handpiece 102 can deliver vibration or other feedback to a user in response to any one or more of a user's input, a state of surgical element 112, and a position of surgical element 112.

Device 100 can include a dial 106 that can be used to rotate shaft 104. Dial 106 can be actuated manually, but dial 106 can also be actuated in an automated fashion, in some embodiments. In some embodiments, dial 106 can be actuated in response to a user's input to touch sensor 108.

As shown, a device 100 can include a battery pack 100. Battery pack 100 can be removeable and/or rechargeable. A device according to the present disclosure can also be connected via a cord or other connector to a power source.

As shown in FIG. 1, a device according to the present disclosure can include a user-visible indicator 114 that is indicative of a state of the device. For example, a device can include an indicator which indicates that the device is powered on. A device can include an indicator that indicates the relative speed at which the surgical element 112 will respond to user input-for example, such an indicator can include a row of 3 LEDs that illuminate such that when the device is in a state wherein the surgical element moves relatively slowly in response to user input, a single one of the 3 LEDs is illuminated; when the device is in a state wherein the surgical element moves at a medium speed in response to user input, two of one of the 3 LEDs are illuminated; and when the device is in a state wherein the surgical element moves at a relatively high speed in response to user input, all three the 3 LEDs are illuminated. In this way, a user can receive visual information concerning the device's state. Likewise, a device can include indicators that are indicative of, for example, a device's charge state, the type of surgical element incorporated into the device, the status of a touch sensor of the device, and other device conditions.

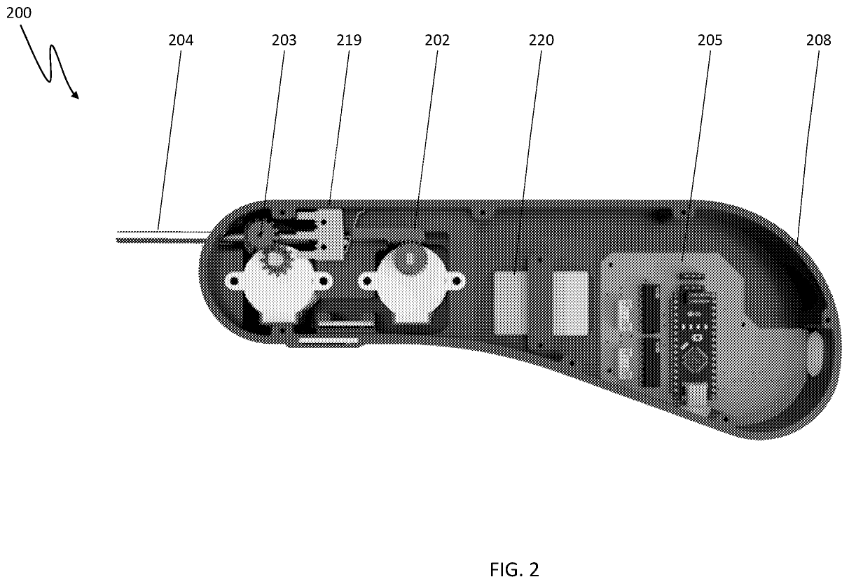

FIG. 2 provides a cutaway view of a device according to the present disclosure. As shown, device 200 can include shaft 204. Actuators 202 and 203 can control movement of the shaft as well as surgical elements (not shown in FIG. 2) that are associated with the shaft. Element 203 can include, for example, bevel gears and a motor, which can operate to control rotation of shaft 204. A motor can be, for example, a stepper motor. A motor can also be, for example, a rotary motor. Motors can be used to control both the rotation and operation (such as opening and closing) of a surgical element. A motor can comprise a rotary encoder so as to allow closed-loop control; additional motors can be added to enable 360-degree motion as well as back-and-forth motion.

As an example, an actuator can operate to advance the shaft, withdraw the shaft, rotate the shaft, effect bending or hinging of the shaft and the like. Likewise, an actuator can effect operation or other motion (for example, advancement and/or rotation) of a surgical element-such as a gripper-that is associated with the shaft. Device 200 can include circuitry 205; such circuitry can include a controller, a processor, and the like. Circuitry 205 can be disposed in base 208 of device 200. As shown, device 200 can include a power cell 220. A power cell can be a battery; a battery can be a rechargeable battery. The battery can be removable. The battery can also be charged in place, for example, by a wired charger. Similarly, a battery can be charged wirelessly. Limit switch 219 can be present; without being bound to any particular theory or embodiment, the limit switch can be used to calibrate motor position. Element 202 can be configured to control opening, closing, or other operation of a surgical element associated with shaft 204. Although not shown, a device according to the present disclosure can include an actuator that effects forward and backward of the shaft, for example to advance the shaft into a patient and to withdraw the shaft from a patient. A device can also include an actuator-not shown-that revolves shaft 204, for example such that the revolution of the shaft scribes a cone-shaped movement path. A device according to the present disclosure can include a button or other controller to effect changes in operational mode. For example, a button can be used to switch between a first mode in which a given movement of one's finger translates into a 90-degree rotation of the shaft and a second mode in which the same finger movement may translate into only a 10-degree rotation of the shaft.

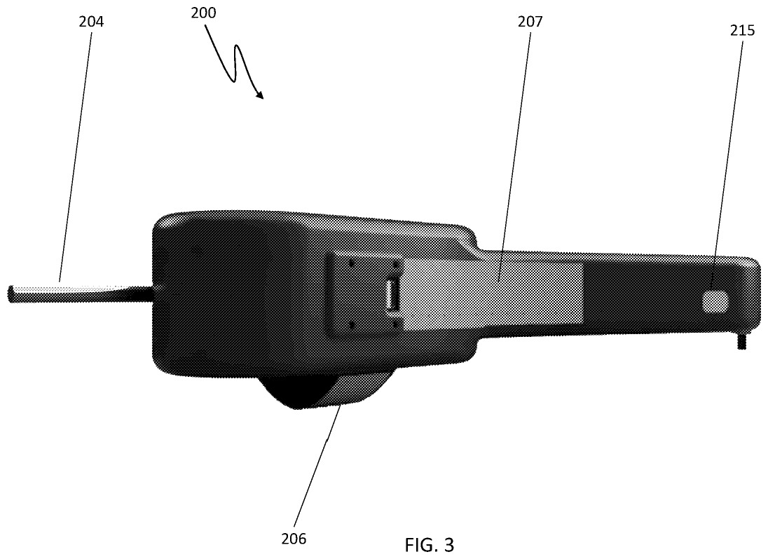

FIG. 3 provides a view of device 200 from a different angle. As shown, device 200 can include a shaft 204. Device 200 can also include sensor 206 and/or sensor 207. A sensor can be a touch sensor; a sensor can also comprise a flexible printed circuit board (or “Flex PCB”), in some embodiments. The shape of a device according to the present disclosure can accommodate movements that a user might make using a traditional handheld device. As shown in FIG. 3, sensors 206 and 207 can be positioned so that a user can engage with both sensors simultaneously. The sensors can be arranged such that a user can engage with one sensor using one finger and engage with the other sensor using another finger or their thumb. This allows a user to achieve fine control over the function of the device; for example a user can use one sensor to control rotation of the shaft and another senor to control operation of a surgical element associated with the shaft. Sensor 206 can be used, for example, to control the rotation of shaft 204; to do so, a user can slide their finger or thumb along sensor 206, and the rotation of shaft 204 can be in response to the sliding of the finger or thumb.

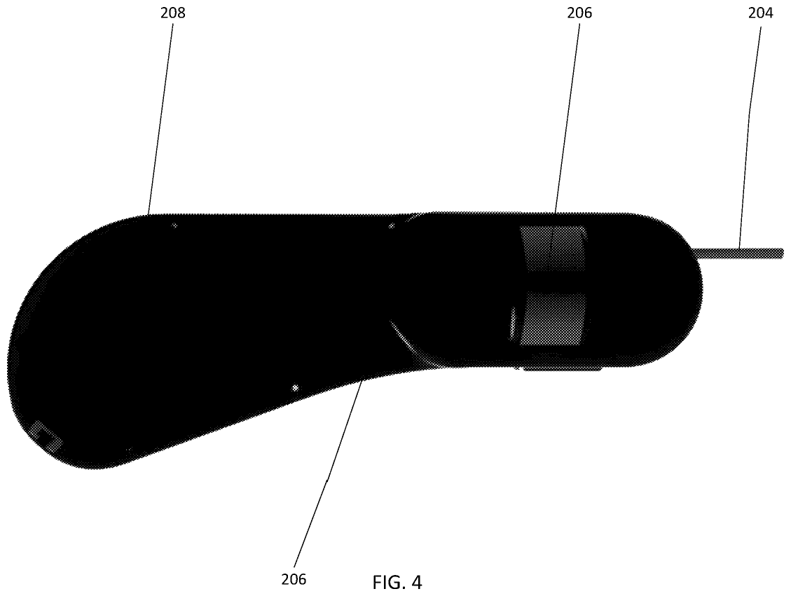

FIG. 4 provides an alternate view of a device according to the present disclosure. As shown, sensor 206 can be present on the side of the device, and sensor 207 can be present on the underside of the device. It should be understood that sensor 207 need not be present, as a mechanical linkage-such as a dial, wheel, or other feature-can be present to allow the user to control shaft 104 or a surgical element associated with the shaft. Likewise, that sensor 206 need not be present, as a mechanical linkage—such as a dial, wheel, or other feature—can be present to allow the user to control shaft 104 or a surgical element associated with the shaft. Cap 215 can cover an opening through which the device can be connected; such an opening can allow a user to connect circuitry within the device to an input or output source, for example a wire connector that allows the user to program or otherwise update instructions residing within the device.

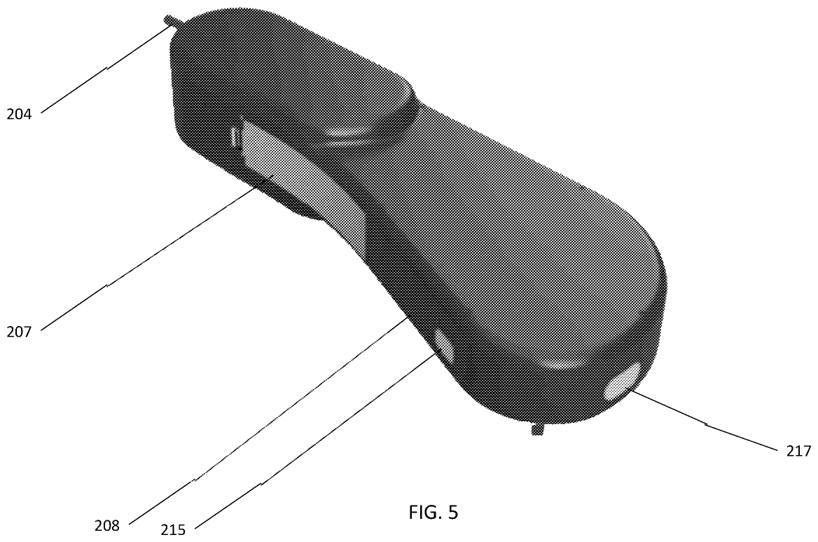

FIG. 5 provides an alternative view of a device according to the present disclosure, showing example relative locations of shaft 104, sensor 207, and base 208. Without being bound to any particular theory or embodiment, the device can be shaped so as to allow a user to hold and manipulate the device with minimal fatigue and discomfort. Cap 217 can be present to cover an opening; such an opening can be used to admit a charger and/or programming cable.

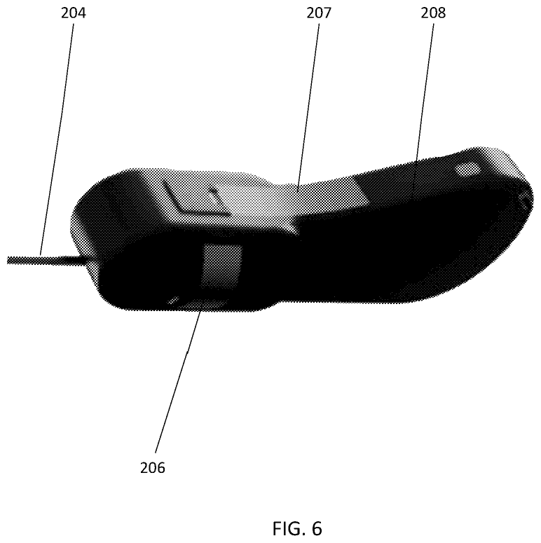

FIG. 6 provides a further alternative view of a device according to the present disclosure, showing example relative locations of shaft 204, sensor 206, sensor 207, and base 208.

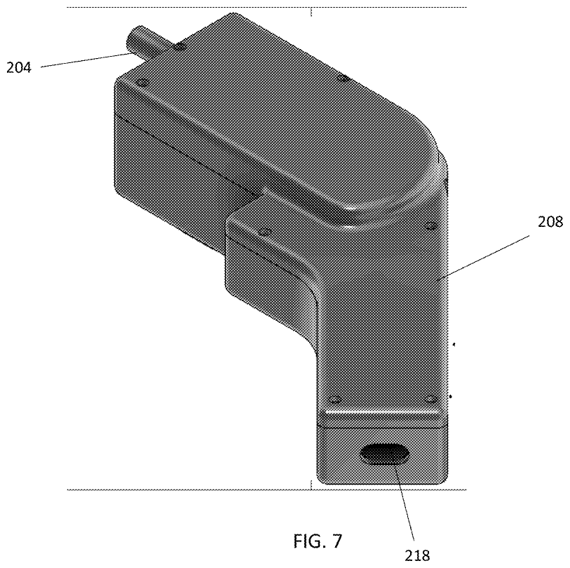

FIG. 7 provides an exterior view of a device according to the present disclosure, showing the relative locations of shaft 204, base 208, and opening 218. Opening 218 can allow access to a charger and/or a programming cable, in some embodiments.

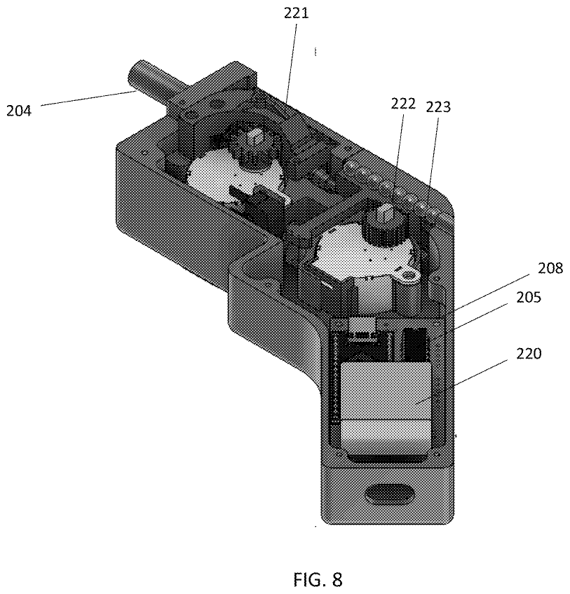

FIG. 8 provides a cutaway view of the device of FIG. 7. As shown, the device can include circuitry 205; such circuitry can include a controller, a processor, and the like. Circuitry 205 can be disposed in base 208 of device 200. As shown, device 200 can include a power cell 220. A power cell can be a battery; a battery can be a rechargeable battery. The battery can be removable. The battery can also be charged in place, for example, by a wired charger.

Actuators 221 and 222 can control movement of the shaft as well as surgical elements (not shown in FIG. 8) associated with the shaft. Actuator 221 can include, for example, bevel gears and a motor, which can operate to control rotation of shaft 204. A motor can be, for example, a stepper motor. A motor can also be, for example, a rotary motor. Likewise, actuator 222 can include, for example, bevel gears and a motor, which can operate to control rotation of shaft 204. A motor can be, for example, a stepper motor. A motor can also be, for example, a rotary motor. One or more ball bearings 223 can be present; without being bound to any particular theory or embodiment, ball bearings can reduce friction along shaft 204 when opening and closing. A radial bearing can be used to reduce friction during shaft rotation.

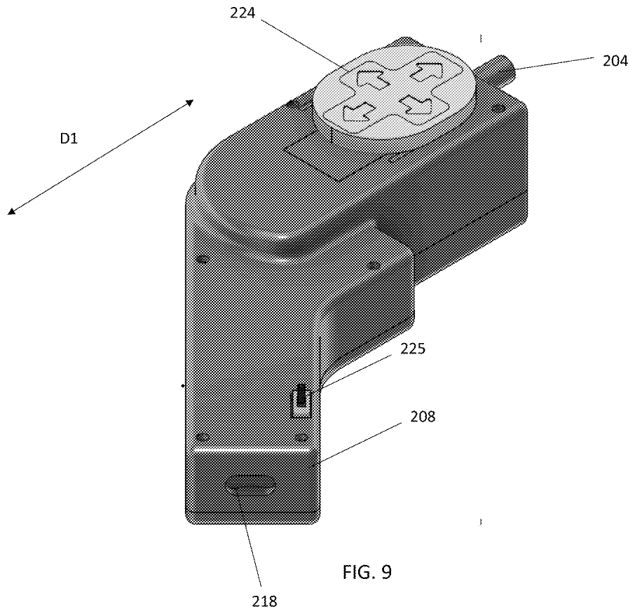

FIG. 9 provides an exterior view of the device of FIG. 7. As shown, the device can include input component 224, which input component can be a multi-button pad. Such a pad can include, for example, two, three, four, five, or more buttons. In an exemplary four-button embodiment, a different button corresponds to each of (i) actuate surgical element in a first manner-such as opening a grasper; (ii) actuating surgical element in a second manner-such as closing a grasper; (iii) rotate the shaft clockwise; and (iv) rotate the shaft counterclockwise. An input component can be moved back and forth along the handle to account for different finger lengths; as an example, input component 224 can be moved along direction DI so as to accommodate a user's preferences. The input component can be arranged such that the device will perform the respective action until the button is released. The buttons can be programmable such that, for example, a user can assign a given function to any one or more of the buttons. In this way, the device can accommodate a range of user preferences, as some users may prefer to have buttons that correspond to rotational action at 12 o'clock and 6 o'clock on the input component shown in FIG. 9, whereas other users may prefer have buttons that correspond to rotational action at 3 o'clock and 0 o'clock on the input component shown in FIG. 9. As shown, the device can include plug 225. Plug 225 can, for example, be a flip switch or other type of switch that can be used to shut power from the device on and off. In some embodiments, plug 225 can be a push button.

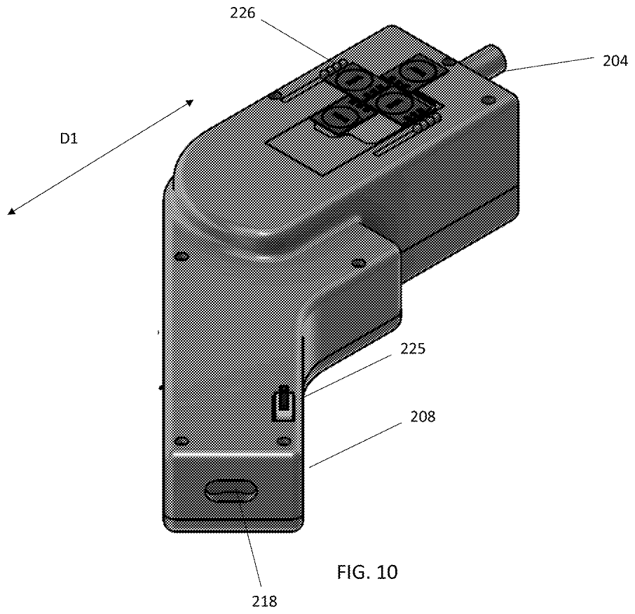

FIG. 10 provides a view of the device of FIG. 9, with input component 224 removed so as to reveal related sensors 226 beneath. As shown, input component 224 can interface with sensors 226; in the non-limiting embodiment shown in FIGS. 9 and 10, each button of input component 224 associates with sensors 226.

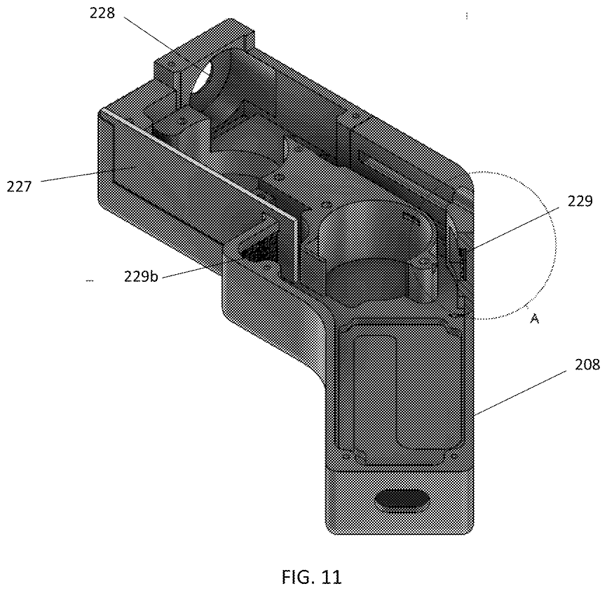

FIG. 11 provides a view of an example device according to the present disclosure, showing the housing of such a device. As shown, base 208 can include an opening 228 through which a shaft (not shown) extends. A slider sensor 227 can be present, which slider sensor can control movement of the shaft and/or surgical element of the device. The slider sensor can be placed, for example, on the top of the device or on the underside of the device as shown in FIG. 11. Sliding a user's finger across the slider sensor can effect movement of the shaft and/or surgical element.

In some embodiments, a toggle switch (not shown) can be present on the device, which toggle switch can be used to switch between open/close and rotational control. As an example, moving the toggle switch can convert the slider switch from controlling rotation of the shaft and/or surgical element to controlling back-and-forth movement of the shaft and/or surgical element. In some embodiments, a further switch (not shown) can be present to control operation of the surgical element, for example to control movement of the surgical element between first and second states, such as between an open state and a closed state. As an example, a first slider switch can be used to translate the shaft and surgical element in a distal direction away from the user and into the subject. A further switch can then be used to open and/or close the surgical element when the surgical element is in the desired position. The first slider switch can also, in some embodiments, be used to rotate the shaft in a clockwise or counterclockwise direction.

A device according to the present disclosure can also include a toggle or other switch used to control an aspect of the device's operation. As an example, a switch can be present and can be used to toggle a slider switch on the device between controlling the open/close status of a grasper associated with the device and the rotation of the grasper. Such a switch can be switchable between two positions, but can also arranged such that when the switch is held down by the user, a slider switch of the device controls the open/close status of the grasper and when the switch is released, the slider switch controls the rotation of the grasper.

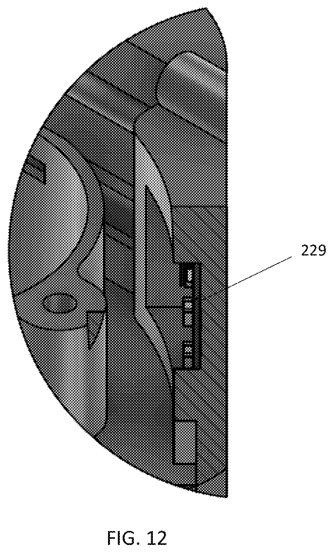

FIG. 12 provides a close view of element 229, which can be a sensor, switch, or other component. A touch sensor can also, for example, be placed beneath the top portion of the device. While such a sensor is pressed, element 227 will control rotational control of the surgical element. When released, element 227 can control open/close control of the surgical element. For example, if element 229 is held, the shaft will rotate clockwise continuously until element 229 is released. Element 229 may also act as a toggle switch that changes what type of motion element 227 controls. Each time element 229 is pressed, element 227 can switch between open/close, rotation, back and forth, and 360-degree control. There can also be an embedded touch sensor—such as element 229b—on the trigger portion of the device nearby to element 227. Sensor 229b can be configured such that pressing the sensor can “snap” the grasper to open/close. As an example, if element 229 is pressed and element 229b is pressed, it can reverse the direction of continuous rotation when element 229 is held. In this way, the disclosed devices can be configured such that actuating one switch or sensor can control the device operation that is controlled by another switch or sensor. In this way.

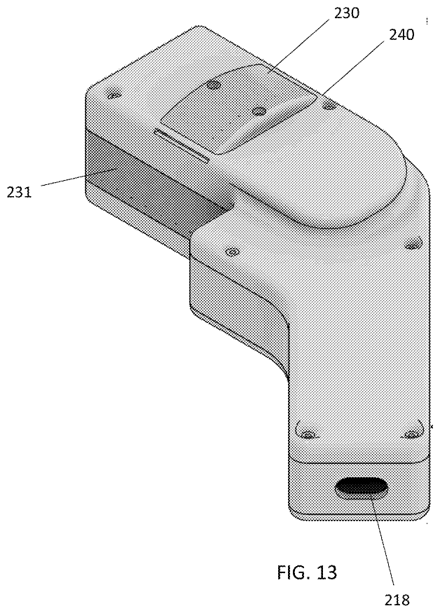

FIG. 13 provides a view of a device according to the present disclosure, which device includes flexible sensors 230 and 231. Such a device can use flexible printed circuit boards (PCBs) to allow for touch control on curved surfaces. These touch sensors can be placed along the sides of the device, to mimic the action of rotating a dial. As the finger slides across the sensors, the shaft of the device (not shown) rotates accordingly. A sensor can be placed along the bottom of the device, which can serve to mimic the curved action of opening and closing seen in a traditional scissors design; as a finger slides across this sensor, a the grasper opens/closes accordingly. As but one example, a sensor could be placed on the top face 240 of the device. Sensors 230 and 231 can also, for example, be embedded directly into the device handle as a printed or laminated conductive layer.

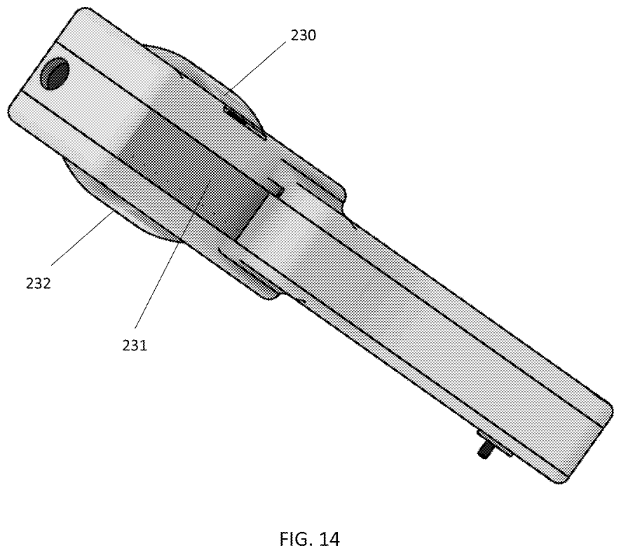

FIG. 14 provides an alternative view of the device of FIG. 13, showing flexible sensors 230, 231, and 232. It should be again understood that the placement of these sensors is illustrative only and should not be understood as limiting sensor placement.

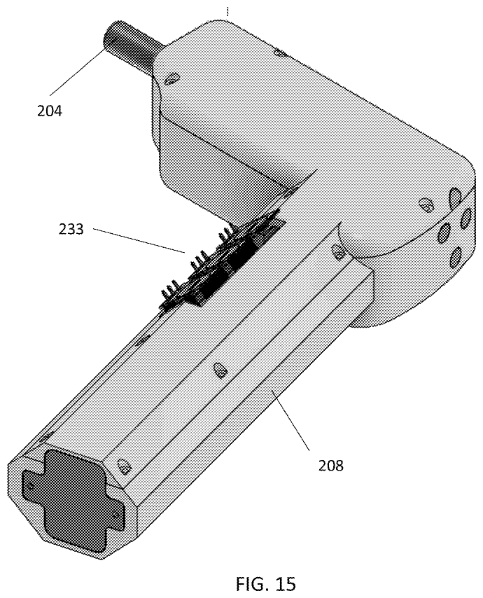

FIG. 15 provides a further embodiment of the disclosed technology. In this embodiment, one can use a vertical set of switches to reduce device size. As shown, one or more switches 233 can be placed on the handle of the device. One of switches can be used to switch between grasping (or translational motion) and rotational control. The other two switches can be used to either grasp or rotate the device depending on the selected state. As an example, actuating the top switch can convert the device from a state in which the second and third switches control rotation of shaft 204 and a state in which the second and third switches control the operation of a surgical element (not shown) that is associated with the shaft, for example, a grasper that is opened and closed via actuation of the second and third switches.

To account for positioning of the device, the device can include a rotary encoder, potentiometer, or other such component associated with the actuator 222 that controls opening and closing of the grasper. As shown, ball bearings 223 can be present to reduce friction along shaft 204.

An actuator can control one or both of rotational motion of the shaft as well as translational motion of the shaft. In some embodiments, one actuator controls rotational motion and another actuator controls translational motion. An actuator can also control operation of the associated surgical element.

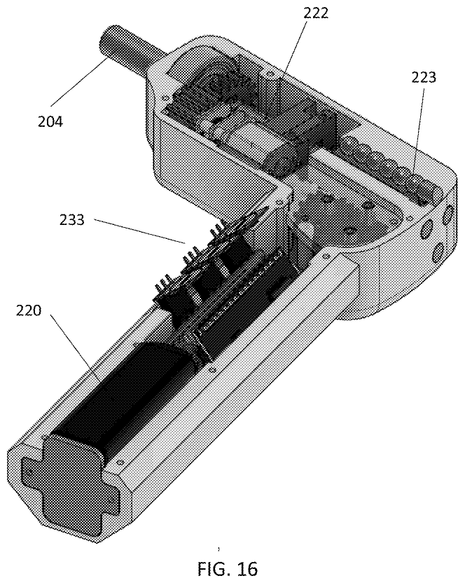

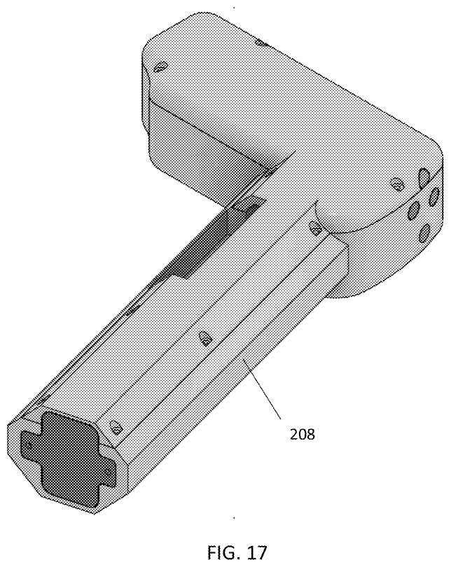

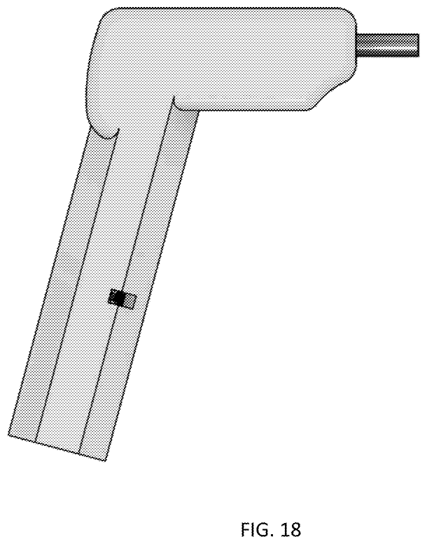

FIG. 17 provides an alternative view of the device according to FIG. 16, showing a fully assembled housing. FIG. 18 provides a side view of the device according to FIG. 17.

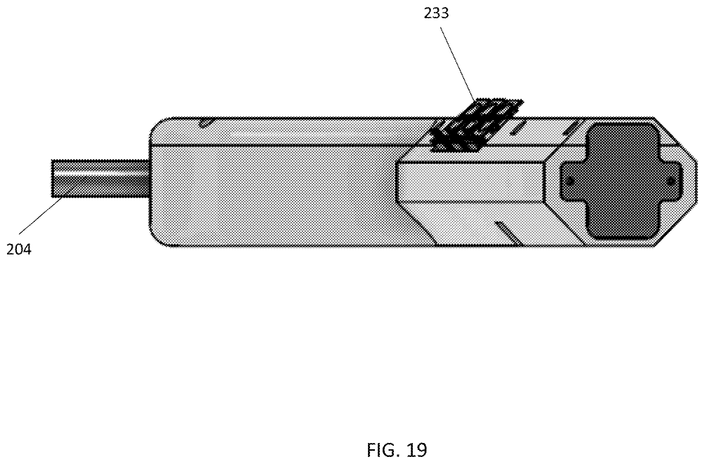

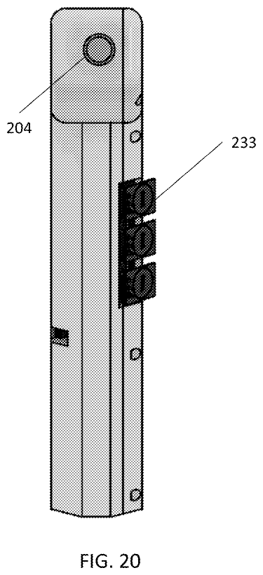

FIG. 19 provides a view of the device according to FIG. 16, looking upwards from the bottom of the device and showing the positioning of switches 233. FIG. 20 provides a view of the device that shows the end of shaft 204 and also the positioning of switches 233 on the handle of the device.

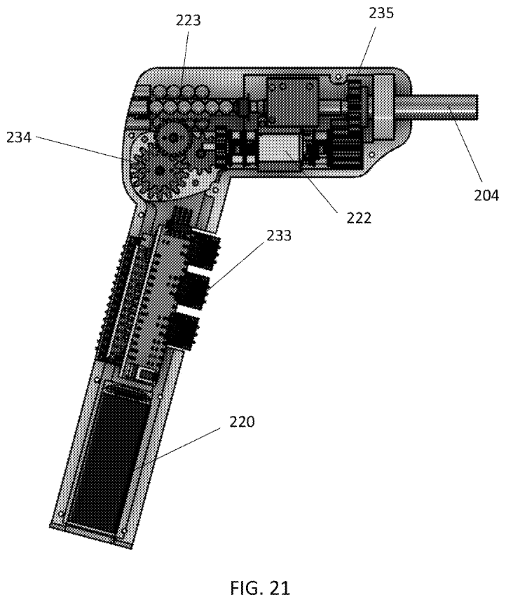

FIG. 21 provides a cutaway view of the device according to FIG. 16. As shown, the device can include power cell 220, which power cell can be removable. The power cell can be recharged via a connector, but can also be recharged wirelessly. Switches 233 can be used to control the activity of actuator 222; as shown, gearsets 234 and 235 can translate the actuator's activity into rotational motion of the shaft 204 as well as into translational motion of the shaft 204 and/or operation of a surgical element (not shown) associated with the shaft. A surgical element can be mounted on the shaft, but can also be associated with the shaft via one or more linkages. As shown, ball bearings 223 can be present to reduce friction on the shaft.

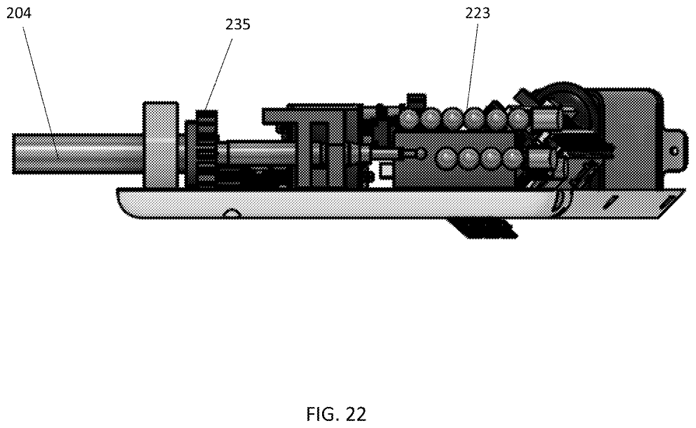

FIG. 22 provides a top cutaway view of the device according to FIG. 21, showing the relative positions of shaft 204, gearset 235, and ball bearings 223.

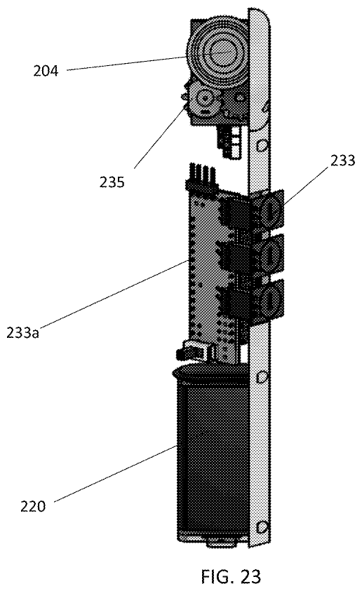

FIG. 23 provides a front cutaway view of the device according to FIG. 21, showing the relative positions of shaft 204, switches 233, gearset 235, and power cell 220.

Also shown is electronics package 233a, which electronics package allows for control of the device using switches 233.

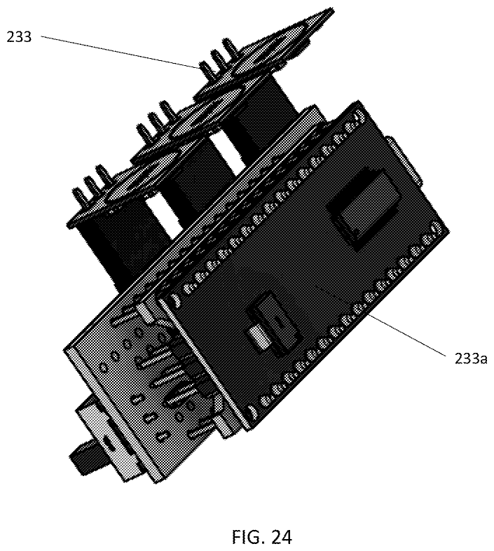

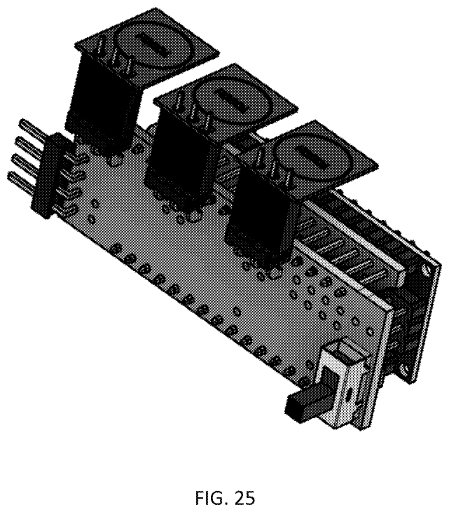

FIG. 24 provides a view of switches 233 and related electronics package 233a. FIG. 25 provides an alternative view of switches 233 and related electronics package 233a.

The following Aspects are illustrative only and do not limit the scope of the present disclosure or the appended claims. Any part or parts of any one or more Aspects can be combined with any part or parts of any one or more other Aspects.

Aspect 1. A surgical device, comprising: a handpiece, the handpiece comprising a touch sensor; a shaft extending from the handpiece and having a distal end; a surgical element disposed generally at the distal end of the shaft; and an actuator, the actuator being controllable by the touch sensor, and the actuator modulating movement of the surgical element.

A surgical device according to the present technology can be, for example, a laparoscopic device. The movement of the surgical element can be, in some embodiments, effected by movement of the shaft. Movement of the shaft, the surgical element, or both, can be, for example, translational, rotational, and the like. The movement can include, as an example, opening or closing of the surgical element.

Aspect 2. The surgical device of Aspect 1, wherein the surgical element comprises any one or more of a jaw, a blade, an arm, and a needle. The surgical element can be characterized as, for example, a grasper or a cutter.

Aspect 3. The surgical device of any one of Aspects 1-2, wherein the shaft is rotatable.

Aspect 4. The surgical device of any one of Aspects 1-3, wherein the actuator comprises any one or more of a motor, a pulley, a gear, a cable, a chain, a spring, a cam, and an elastomer. A motor can be a stepper motor, servo motor and/or a DC motor, for example. An actuator can also be a linear actuator.

Aspect 5. The surgical device of any one of Aspects 1-4, wherein the surgical device is configured such that a touch on the touch sensor modulates the actuator to effect a movement of the surgical element. It should be understood that in some embodiments, a touch on the touch sensor modulates the actuator to effect a movement of the shaft, which movement of the shaft can in some embodiments give rise to movement of the surgical element, as described elsewhere herein.

Aspect 6. The surgical device of Aspect 5, wherein the touch is a sliding touch along a distance on the touch sensor.

Aspect 7. The surgical device of Aspect 6, wherein a movement of the surgical element is proportional to the distance.

Aspect 8. The surgical device of Aspect 6, wherein the sliding touch is in a first direction along the touch sensor and wherein the movement of the surgical element is in a first direction.

Aspect 9. The surgical device of Aspect 6, wherein the sliding touch is in a second direction along the touch sensor and wherein the movement of the surgical element is in a second direction. The first direction and second direction can be opposite to one another, although this is not a requirement. The first and second direction can be parallel to one another, in some instances. The first and second direction can be at an angle to one another, in some instances.

Aspect 10. The surgical device of Aspect 5, wherein the touch is a tap.

Aspect 11. The surgical device of any one of Aspects 1-4, wherein the tap modulates a degree of movement of the surgical element.

Aspect 12. A surgical device, comprising: a handpiece; a surgical element; and a control train configured to control a movement of the surgical element in response to a user's touch on a touch sensor of the control train.

A control train can include, for example, one or more microprocessors, which microprocessors can be comprised in microcontrollers. As but one example, a device according to the present disclosure can include an ATmega328P microcontroller, which interprets the signals from the touch sensors and relays such signals to the actuators of the device. This microcontroller can also be switched out for an ESP32, allowing for wireless communication with companion devices. It should be understood that a microcontroller in the disclosed devices can be either wired or wireless.

As described elsewhere herein, a device according to the present disclosure can include one or more limit switches. Such switches can be placed within the device so as to calibrate the initial position of the surgical element, such as a grasper.

Aspect 13. The surgical device of Aspect 12, wherein the surgical element comprises any one or more of a jaw, a blade, an arm, and a needle.

Aspect 14. The surgical device of any one of Aspects 12-13, wherein (1) the movement is in response to a sliding touch on the touch sensor, (2) the movement is in response to a tap touch on the touch sensor, or both (1) and (2).

Aspect 15. The surgical device of Aspect 14, wherein the direction of movement is in response to a sliding touch on the touch sensor and wherein the degree of movement is modulated in response to a tap on the touch sensor.

Aspect 16. The surgical device of Aspect 14, wherein the degree of movement is modulated in response to a number of recorded taps on the touch sensor.

Aspect 17. A method, comprising: in response to a touch on a touch sensor of a surgical device, controlling any one or more of a direction of motion or a degree of motion of a surgical element of the surgical device. Such a device can be, for example, a device according to the present disclosure.

Aspect 18. The method of Aspect 17, wherein the direction of motion of the surgical element is in response to a sliding touch on the touch sensor.

Aspect 19. The method of any one of Aspects 17-18, wherein the degree of motion of the surgical element is in response to a tap on the touch sensor.

Aspect 20. The method of any one of Aspects 17-19, wherein the surgical element comprises any one or more of a jaw, a blade, an arm, and a needle.

What is claimed:

1. A surgical device, comprising:
a handpiece,
the handpiece comprising a touch sensor;
a shaft extending from the handpiece and having a distal end;
a surgical element disposed generally at the distal end of the shaft; and
an actuator,
the actuator being controllable by the touch sensor, and
the actuator modulating movement of the surgical element.

2. The surgical device of claim 1, wherein the surgical element comprises any one or more of a jaw, a blade, an arm, and a needle.

3. The surgical device of claim 1, wherein the shaft is rotatable.

4. The surgical device of claim 1, wherein the actuator comprises any one or more of a motor, a pulley, a gear, a cable, a chain, a spring, a cam, and an elastomer.

5. The surgical device of claim 1, wherein the surgical device is configured such that a touch on the touch sensor modulates the actuator to effect a movement of the surgical element.

6. The surgical device of claim 5, wherein the touch is a sliding touch along a distance on the touch sensor.

7. The surgical device of claim 6, wherein a movement of the surgical element is proportional to the distance.

8. The surgical device of claim 6, wherein the sliding touch is in a first direction along the touch sensor and wherein the movement of the surgical element is in a first direction.

9. The surgical device of claim 6, wherein the sliding touch is in a second direction along the touch sensor and wherein the movement of the surgical element is in a second direction.

10. The surgical device of claim 5, wherein the touch is a tap.

11. The surgical device of claim 10, wherein the tap modulates a degree of movement of the surgical element.

12. A surgical device, comprising:
a handpiece;
a surgical element; and
a control train configured to control a movement of the surgical element in response to a user's touch on a touch sensor of the control train.

13. The surgical device of claim 12, wherein the surgical element comprises any one or more of a jaw, a blade, an arm, and a needle.

14. The surgical device of claim 12, wherein (1) the movement is in response to a sliding touch on the touch sensor, (2) the movement is in response to a tap touch on the touch sensor, or both (1) and (2).

15. The surgical device of claim 14, wherein a direction of movement is in response to a sliding touch on the touch sensor and wherein a degree of movement is modulated in response to a tap on the touch sensor.

16. The surgical device of claim 14, wherein a degree of movement is modulated in response to a number of recorded taps on the touch sensor.

17. A method, comprising:
in response to a touch on a touch sensor of a surgical device, controlling any one or more of a direction of motion or a degree of motion of a surgical element of the surgical device.

18. The method of claim 17, wherein the direction of motion of the surgical element is in response to a sliding touch on the touch sensor.

19. The method of claim 17, wherein the degree of motion of the surgical element is in response to a tap on the touch sensor.

20. The method of claim 17, wherein the surgical element comprises any one or more of a jaw, a blade, an arm, and a needle.
